(19) United States
(12) Patent Application Publication
(10) Pub. No.: US 2025/0032290 A1
(43) Pub. Date: Jan. 30, 2025

(54) DELIVERY SYSTEMS FOR ENDOLUMINAL PROSTHESES AND METHODS OF USE

(71) Applicant: Silk Road Medical, Inc., Sunnyvale, CA (US)

(72) Inventors: Peter Soliman, Sunnyvale, CA (US); Karl Halden, Sunnyvale, CA (US); Huy Tran, Sunnyvale, CA (US); Akshay Hulasare, Sunnyvale, CA (US)

(21) Appl. No.: 18/717,955

(22) PCT Filed: Dec. 6, 2022
(86) PCT No.: PCT/US2022/080992

§ 371 Date: Jun. 7, 2024

Related U.S. Application Data
(60) Provisional application No. 63/287,282, filed on Dec. 8, 2021.

(51) Int. Cl. A61F 2/966; A61F 2/95

(52) U.S. Cl. CPC A61F 2/966; A61F 2/9517

(57) ABSTRACT

An endoluminal prostheses delivery system having an outer sheath with a distal end region, a proximal end region and a lumen extending along a longitudinal axis between the proximal end region and the distal end region. An inner member extends through the lumen of the outer sheath, at least a portion of the inner member configured to support an expandable device within the lumen near the distal end region of the outer sheath. A release mechanism is configured to move at least one of the outer sheath and the inner member a distance in a controlled manner to break any friction between the expandable device and the outer sheath prior to deployment of the expandable device from the lumen. Related systems, devices, and methods are provided.

### CROSS-REFERENCE TO RELATED APPLICATIONS

This application claims the benefit of priority under 35 U.S.C. § 119 (e) to Provisional Patent Application Ser. No. 63/287,282, filed Dec. 8, 2021. The disclosure of the provisional application is incorporated by reference in its entirety.

### BACKGROUND

The present disclosure relates to tubular endoluminal prostheses, such as stents, stent-grafts, and the like. More particularly, described are delivery systems and methods for their use to accurately and safely deploy endoluminal prostheses within the lumen of a body, particularly within the vascular system for the treatment of aneurysms, stenosis, and the like, that incorporate mechanisms to release static friction and slack prior to deployment of the prostheses within the vessel.

Stent-grafts and self-expanding stents are resilient structures biased to expand against the surrounding luminal wall. To deliver these structures to a target location, they are tightly compressed within a delivery system. “Pin-and-pull” stent delivery systems generally include an inner support catheter (e.g., a tube or a rod) and an outer sheath. The outer sheath longitudinally translates relative to the inner support catheter to deploy the expandable structure that was previously compressed inside the distal end region of the outer sheath. The deployment involves “pinning” the inner support catheter relative to the patient and “pulling” back on the outer sheath exposing the expandable structure so that it can expand outward against a vessel.

These delivery systems, in order to reach the target stenosis or aneurysm, typically must maneuver within highly tortuous anatomy. After navigating several curves to reach the target site, the catheter system meets resistance and can store tension proximal to the distal tip. As the distal tip of the delivery system navigates a straight segment of a vessel and enters a bend, an amount of tension can get stored. Upon exiting the bend and entering another straight segment, the tension can get released and propel the entire system forward creating a “jump”. The system can develop slack or buckle due to downward forces pushing on the outer sheath. Stored tension in the sheath can be problematic because it can propel the entire system distally once the tension is released, such as when unsleeving the sheath from the self-expanding stent for deployment at the target location. The movement of the distal end of the sheath upon release of the stored tension as the proximal end is retracted for deployment can result in the stent being deployed so that it misses the target site during unsheathing.

The forces between the outer sheath of the delivery system and the expanding structure in combination with the tension built up within the sheath during navigation to the target site can result in a sharp rise in deployment forces at initiation of deployment of the expanding structure followed by a quick drop-off in deployment forces for the remainder of deployment. The change in deployment forces can negatively impact deployment accuracy at the implantation site. The highest deployment force occurs in the initial stage of outer sheath retraction. Once the initial friction between the sheath and the compressed expandable structure is overcome, the force for deployment of the structure drops off almost instantaneously. The result can be an expandable structure that jumps out of the delivery system and/or inadvertent motion by the user that causes the expanding structure to be deployed inaccurately relative to the target site.

Conventional pin-and-pull systems do not provide the user with mechanisms to control deployment speed or force. Some stent delivery systems incorporate mechanisms to control deployment of the expandable structure, for example, mechanical handles with thumbwheels that retract the outer sheath incrementally or stops that prevent inadvertent jumping of the stent out of the sheath. While these mechanical systems provide control and improve accuracy they are tedious to use and do not provide the quick and convenient deployment of the pin-and-pull system.

Accordingly, a need exists for quick and convenient deployment of expandable structures that are also safe, accurate, and controlled.

### SUMMARY

In an aspect, provided is an endoluminal prostheses delivery system having an outer sheath with a distal end region, a proximal end region and a lumen extending along a longitudinal axis between the proximal end region and the distal end region; an expandable device; an inner member extending through the lumen of the outer sheath, at least a portion of the inner member configured to support the expandable device within the lumen near the distal end region of the outer sheath; and a release mechanism configured to move at least one of the outer sheath and the inner member an initial distance in a controlled manner to break friction between the expandable device and the outer sheath prior to deployment of the expandable device from the lumen.

The release mechanism can be configured to retract the outer sheath the distance. The release mechanism can be configured to advance the inner member the distance. The distance is about 5 mm up to about 1 cm. The release mechanism can include an actuator configured to be toggled between at least a first position and at least a second position. The actuator can be toggled by rotation around longitudinal axis of the outer sheath. The release mechanism can further include a threaded internal component coupled to a proximal end region of the inner member. The threaded internal component can be in threaded engagement with a corresponding thread on an internal surface of the actuator. Toggling the actuator around the longitudinal axis from the first position to the second position while the inner member is held fixed and the outer sheath is not held fixed can retract the outer sheath proximally as the corresponding thread of the actuator translates along the threaded internal component. Toggling the actuator around the longitudinal axis from the first position to the second position while the proximal end region of the outer sheath is held fixed and the inner member is not held fixed can advance the inner member distally as the threaded internal component translates along the corresponding thread of the actuator.

The actuator can further include a projection on an outer surface of the actuator configured to be received against or within a first stop on a proximal end region of the outer sheath. The first stop can provide tactile and/or auditory feedback regarding position of the actuator relative to the first stop. The actuator can include a projection and the first stop can include a first surface feature projecting a distance radially outward from an outer surface of the outer sheath and a second surface feature projecting a second distance radially outward from the outer surface of the outer sheath, the first surface feature projecting further than the second surface feature such that the projection on the actuator can slide over the second surface feature and is prevented from sliding over the first surface feature. Receipt of the projection between the first and second surface features can provide tactile and/or auditory feedback regarding position of the actuator relative to the first stop. The system can further include one or more markings on the outer surface of the outer sheath relative to the first stop providing information regarding actuator position. A second stop can be on the proximal end region of the outer sheath located less than 360 degrees around the longitudinal axis relative to the first stop. The actuator can be toggled by rotation around an axis orthogonal to the longitudinal axis of the outer sheath.

The release mechanism can further include a cam body positioned within a cam hub coupled to a proximal end region of the outer sheath. The cam body can have an elliptical shape configured to project through an opening in the cam body and into the lumen of the proximal end region of the outer sheath. The elliptical shape of the cam body can include a plurality of teeth. The inner member can include one or more surface features sized and spaced to engage with the plurality of teeth on the cam body. The cam body and the inner member come into contact with one another within a region of the lumen of the outer sheath. The cam body can pinch the inner member against an inner wall of the outer sheath to lock and/or drive the inner member upon rotation of the cam body. Toggling the actuator can include moving the actuator relative to the lumen between a first locked position, a second released position, and a third deployment position. The first locked position can include the actuator fully cammed over so that the inner member is pinched between the cam body and the lumen effecting a lock. Rotating the cam body around the axis away from the first locked position toward the second released position while the inner member is not fixed can drive the inner member forward the distance. Rotating the cam body around the axis away from the first locked position toward the second released position while the inner member is kept fixed can retract the outer sheath the distance in a proximal direction relative to the inner member. Rotating the cam body around the axis away from the second released position toward the third deployment position fully withdraws the cam body from the lumen of the outer sheath. The cam body and/or the cam hub can incorporate one or more detents to provide tactile and/or audible feedback regarding relative position of the actuator.

In some variations, one or more of the following can optionally be included in any feasible combination in the above methods, apparatus, devices, and systems. More details are set forth in the accompanying drawings and the description below. Other features and advantages will be apparent from the description and drawings.

### BRIEF DESCRIPTION OF THE DRAWINGS

These and other aspects will now be described in detail with references to the following drawings. Generally speaking the figures are exemplary and are not to scale in absolute terms or comparatively but are intended to be illustrative. Relative placement of features and elements is modified for the purpose of illustrative clarity.

FIG. 1 shows an implementation of a delivery system having a release mechanism;

FIG. 2A is a side view of an implementation of a delivery system having a threaded rotator release mechanism;

FIG. 2B is a cut-away side view of the delivery system of FIG. 2A;

FIG. 2C is a side view of the delivery system of FIG. 2A;

FIG. 3A shows an implementation of a delivery system having a cam handle release mechanism;

FIG. 3B shows the sheath hub of the cam handle release mechanism of FIG. 3A having the push rod extending therethrough;

FIGS. 4A-4B are perspective views of a sheath hub of the cam handle release mechanism of FIG. 3A;

FIG. 4C is a cross-sectional view of the sheath hub of FIG. 4A;

FIG. 4D is a perspective view of a cam body for use with the cam handle release mechanism of FIG. 3A;

FIGS. 5A-5C are process schematic views of the cam handle release mechanism of FIG. 3A in locked, released, and deployment positions, respectively.

### DETAILED DESCRIPTION

The present disclosure relates to tubular endoluminal prostheses, such as stents, stent-grafts, and the like. More particularly, described are improved delivery systems and methods for their use to accurately and safely deploy endoluminal prostheses within the lumen of a body, particularly within the vascular system for the treatment of aneurysms, stenosis, and the like, that incorporate a handle having mechanisms to release static friction and slack prior to deployment of the prostheses within the vessel.

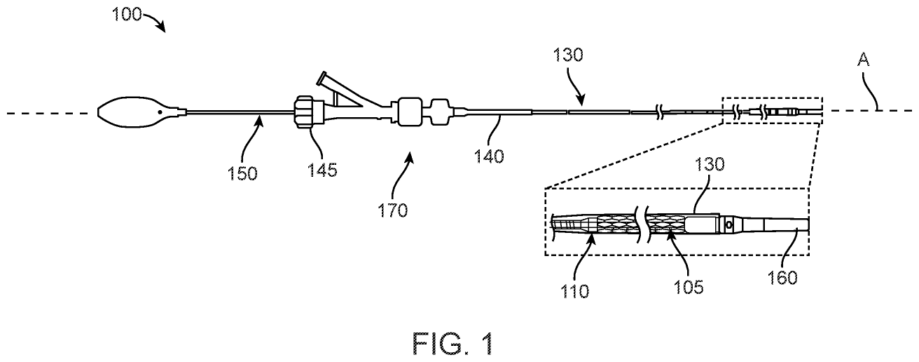

FIG. 1 illustrates a delivery system 100 having an inner catheter 110 disposed within an outer sheath 130 that houses an expandable device 105. A proximal end region of the outer sheath 130 can be coupled to a sheath hub 140 positioned distal to a hemostasis valve 145. The hemostasis valve 145 can be a part of a y-arm connector as is known in the art. The inner catheter 110 can be longitudinally and rotationally fixed to a push rod 150, such as a stainless steel hypotube at its proximal end region. The inner catheter 110 extends through the outer sheath 130 via the hemostasis valve 145. A nose cone 160 can be coupled to a distal end region of the inner catheter 110 that projects distally of the outer sheath 130 and the device 105 contained within the outer sheath 130. The delivery system 100 can additionally incorporate a release mechanism 170. The release mechanism 170 can function in one of two ways. In a first method, the release mechanism 170 involves the outer sheath 130 being retracted proximally a set distance while the inner catheter 110 is pinned. In this method, the device 105 does not move relative to where it is positioned in the anatomy. The release mechanism 170 functions to initially retract the outer sheath 130 proximally a set distance and in a controlled manner to relieve slack built up within the stent delivery system 100 and to break any friction between the device 105 and the outer sheath 130 prior to deployment of the device 105 from the system 100. The reduction or elimination of the built-up friction, tension, and/or slack prior to deployment of the expandable device improves accuracy and safety of the device deployment. Alternatively, the release mechanism 170 involves the inner catheter 110 being moved distally the set distance while the outer sheath 130 is pinned, for example, by a user gripping the sheath hub 140. In this method, the device 105 would move distally the set distance (assuming the push rod 150 is in full contact with the proximal end of the device 105). The release mechanism 170 will be described in more detail below.

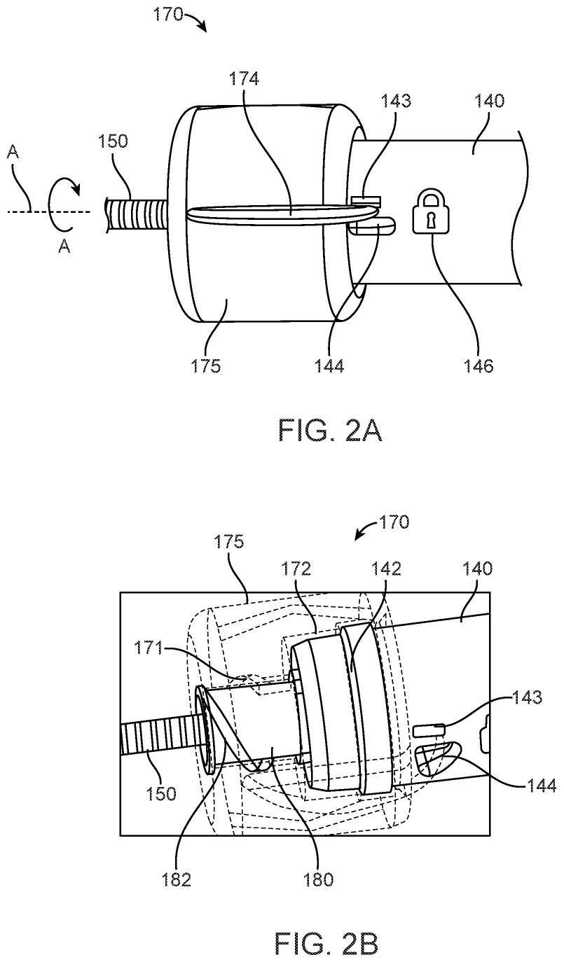

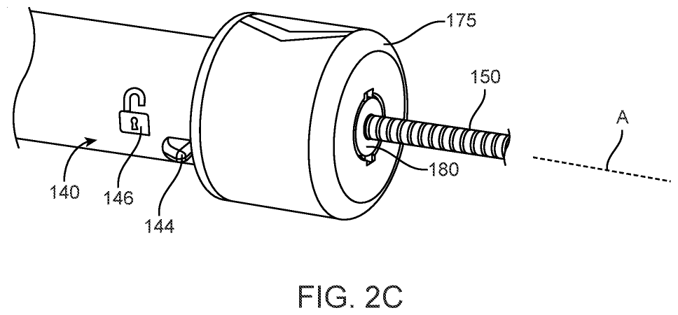

FIGS. 2A-2C illustrate an implementation of a release mechanism 170 for use with the delivery system 100 for deployment of an expandable device 105. The release mechanism 170 can include an actuator 175 configured to move the sheath hub 140 relative to the push rod 150. The actuator 175 can be toggled between a first position and a second position. The first position of the actuator 175 can be a “locked” position where the sheath hub 140 is in its home position relative to the push rod 150. The actuator 175 can be toggled, such as by rotating it a fixed number of degrees around the axis A of the sheath 130, for example, 180 degrees from the first position to the second position. The second position of the actuator 175 can be an “unlocked” position where the sheath hub 140 is translated along its longitudinal axis A in a proximal direction a fixed distance. The fixed distance the sheath hub 140 moves upon toggling the actuator 175 can be about 5 mm up to about 1 cm. The design of the system can accommodate no sheath movement (i.e., 0 mm) as well up to full deployment (e.g., 20-50 mm). The distance of full deployment can be designed to accommodate a length of the implanted intended for delivery. The sheath hub 140 is coupled to a proximal end region of the outer sheath 130. Retraction of the sheath hub 140 the fixed distance by toggling the actuator 175 into the unlocked position moves the distal end of the outer sheath 130 the fixed distance relative to the expandable device 105 compressed on the inner catheter 110. The retraction of the outer sheath 130 breaks any built-up friction that may exist between the device 105 and the outer sheath 130.

The actuator 175 can be a barrel-shaped component having an inner dimension sized to mate with an outer dimension of the sheath hub 140. FIGS. 2A-2B show the proximal end region of the sheath hub 140 mated with the distal end region of the actuator 175. The hub 140 can incorporate a feature 142 on its outer surface configured to engage with a corresponding feature 172 on the inner surface of the actuator 175. For example, the hub 140 and the actuator 175 can couple together via threads or other connection features.

The actuator 175 also incorporates a threaded internal component 180 within its inner dimension. The internal component 180 and the actuator 175 are configured to be in threaded engagement with one another so that as the actuator 175 toggles around the longitudinal axis A the internal component 180 is urged axially along the longitudinal axis A. The internal component 180 can have a thread 182 on an outer surface that is engaged with a corresponding thread 171 on an internal surface of the actuator 175. Alternatively, one component can have a single pin that is configured to engage and travel within a corresponding thread of the other component. For example, the internal component 180 can have a pin rather than the thread 182 where the pin engages with and slides within the thread 171 on the internal surface of the actuator 175. The internal component 180 can also include an inner diameter sized to receive the push rod 150 of the inner catheter 110 or another hypotube or push rod connected to the push rod 150 of the delivery system 100 so that the internal component 180 is fixed to the push rod 150. As the actuator 175 is toggled around the longitudinal axis A from the locked position to the unlocked position, the push rod 150 can be pinned by a user to prevent it from moving so that the internal component 180 will move axially relative to the actuator 175. The actuator 175 and internal component 180 can move apart either by the actuator 175 moving proximally if a standard pin/pull unlock, or the internal component 180 moves distally if no pinning during unlocking with the actuator 175. For example, the actuator 175 can translate proximally along the thread 182 of the internal component 180 retracting the hub 140 a distance along the longitudinal axis A and thereby retracting the sheath 130 that same distance. It should be appreciated that any number of threaded rotating mechanisms are considered herein to toggle between the two positions and achieve proximal retraction of the outer sheath 130. Also, in some implementations the push rod 150 is not pinned and a user holds the sheath hub 140 fixed while toggling the actuator 175. The internal component 180 and push rod 150 can translate distally along the thread 171 of the actuator 175 and thereby at the distal end the device 105 and tip move distally that same distance.

Again with respect to FIGS. 2A-2C, the actuator 175 can include a projection 174 projecting radially outward from the outer surface of the barrel-shaped component. The projection 174 can be received against or within a corresponding surface feature or stop 144 on the proximal end region of the sheath hub 140. For example, FIG. 2A shows the actuator 175 positioned relative to the hub 140 in the “locked” position. The sheath hub 140 is in its distal-most position relative to the device 105. The projection 174 on the actuator 175 can abut against the stop 144 on the sheath hub 140 allowing rotation around the longitudinal axis of the sheath 130 only in a single direction relative to the stop 144. FIG. 2C shows the opposite side of the sheath hub 140 as what is shown in FIG. 2A. This opposite side of the sheath hub 140 can include a second stop 144 positioned around a circumference of the barrel-shaped component (e.g., about 180 degrees) away from the first stop 144 for the “unlocked” position. The actuator 175 can be toggled around the longitudinal axis A of the sheath in the direction of arrow A away from the “locked” position stop 144 visible in FIG. 2A towards the “unlocked” position stop 144 visible in FIG. 2C. The projection 174 on the actuator 175 abuts against the stop 144 in the “locked” position allowing rotation in the direction of arrow A and preventing rotation in the direction opposite of arrow A. The projection 174 again abuts against the stop 144 in the “unlocked” position preventing further rotation in the direction of arrow A and allowing rotation in the direction opposite of arrow A. The first stop 144 for the “locked” position and the second stop 144 for the “unlocked” position prevent the actuator 175 from rotating the full 360 degrees around the longitudinal axis A and instead allow for the actuator to toggle between two positions—the locked and unlocked positions. The stops 144 shown in FIGS. 2A and 2C are positioned on the outer sheath to allow 180 degree rotation in a first direction and another 180 degree rotation in a second, opposite direction. The degree rotation can vary, but is generally less than the full 360 degrees around the axis A.

One or both of the stops 144 can provide tactile and/or auditory feedback regarding the position of the actuator 175 relative to the stops 144. The stop 144 in FIG. 2A shows a smaller projection 143 positioned a distance away from stop 144 that upon receiving the projection 174 of the actuator 175 therebetween can provide a “click” or “snap” noise or feel for the user to understand the actuator 175 is in the locked position. The stop 144 can project a distance radially outward from an outer surface of the outer sheath that is greater than a distance the smaller projection 143 projects. The projection 174 of the actuator 175 can slide over the smaller projection 143, but is prevented from sliding over the larger sized stop 144. One or both of the stops 144 on the outer sheath can incorporate pairs of larger and smaller sized projections to provide feedback to the user.

The sheath hub 140 can additionally incorporate one or more markings 146 providing the user with information regarding the position of the actuator 175. A first marking 146 can be positioned at the first position to provide the user with information about the actuator 175 being in the first “locked” position and a second marking 146 can be positioned at the second position to provide the user with information about the actuator 175 being in the second “unlocked” position. The markings 146 can have a design indicative of the operational state such as a lock in a locked state and a lock in an unlocked state or can incorporate the words “locked” and “unlocked” or some other word indicating the operational state of the device.

Once the greatest force is overcome in the initial stages of outer sheath 130 withdrawal, which is relieved in a controlled manner by the release mechanism 170, the longitudinal retraction of the outer sheath 130 can be performed per conventional pin-and-pull technique without risk of handle jerk or slippage or jumping of the stent out of the delivery system 100.

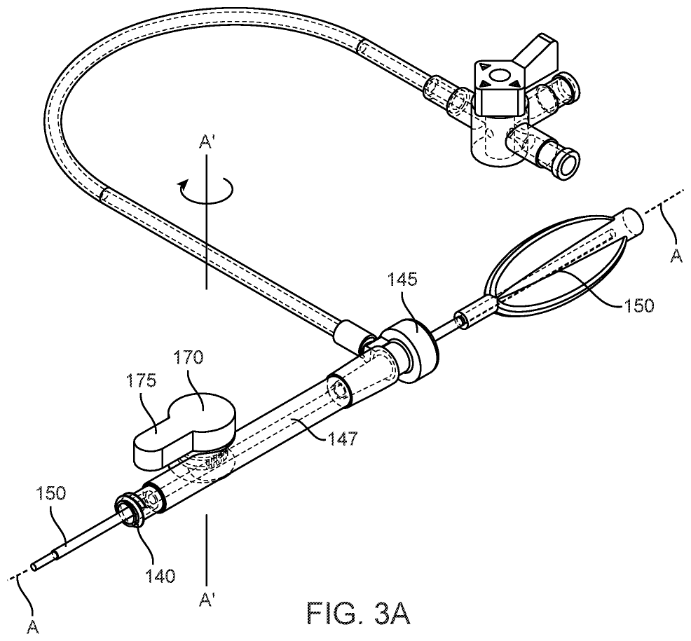

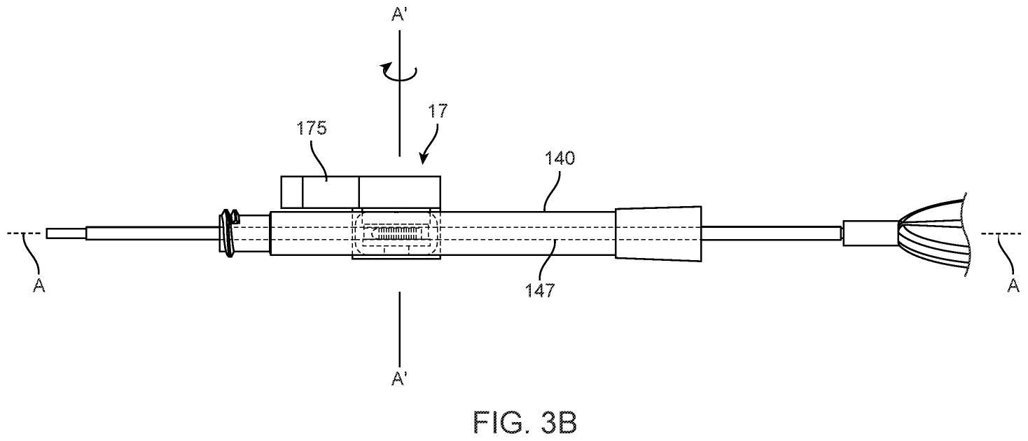

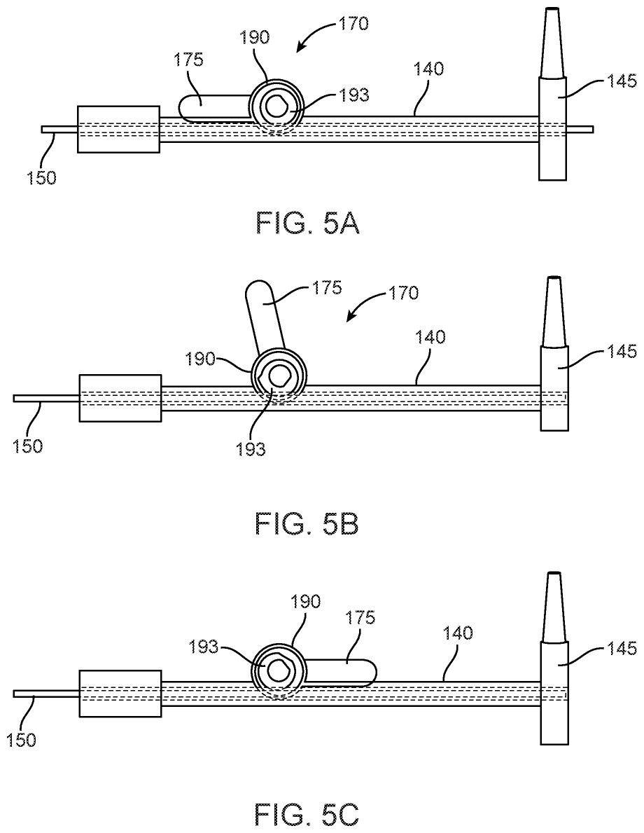

FIGS. 3A-3B show another implementation of a release mechanism 170 for use with a delivery system 100. FIGS. 3A-3B show the delivery system 100 without the outer sheath 130 coupled to the sheath hub 140. The push rod 150 extends through the hemostasis valve 145 and within a lumen 147 of the sheath hub 140. As with the other implementations, the release mechanism 170 can incorporate an actuator 175 configured to move the sheath hub 140 relative to the push rod 150. The actuator 175 is configured to rotate around an axis A′ that is orthogonal to the longitudinal axis A of the sheath 130 to achieve proximal retraction of the sheath 130. The actuator 175 can be rotated around axis A′ about 180 degrees (see e.g., FIGS. 5A-5C) although the degree of rotation can vary to deploy the entire stent. For example, continued rotations of the actuator 175 are also possible such that the motion can be between 0 mm all the way to full deployment of the expandable device 105.

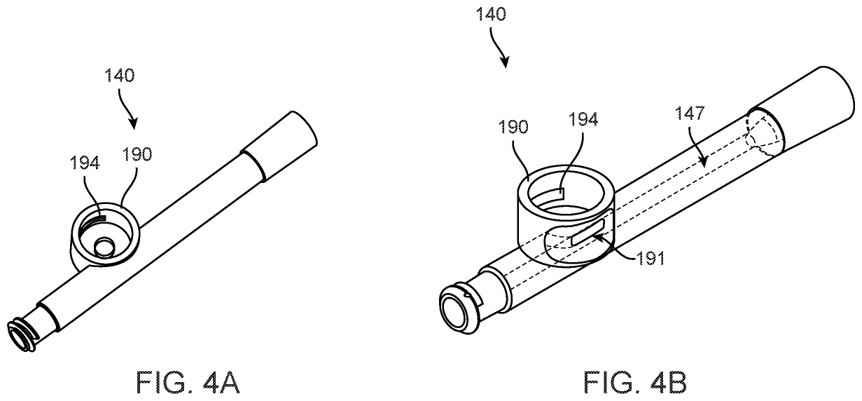

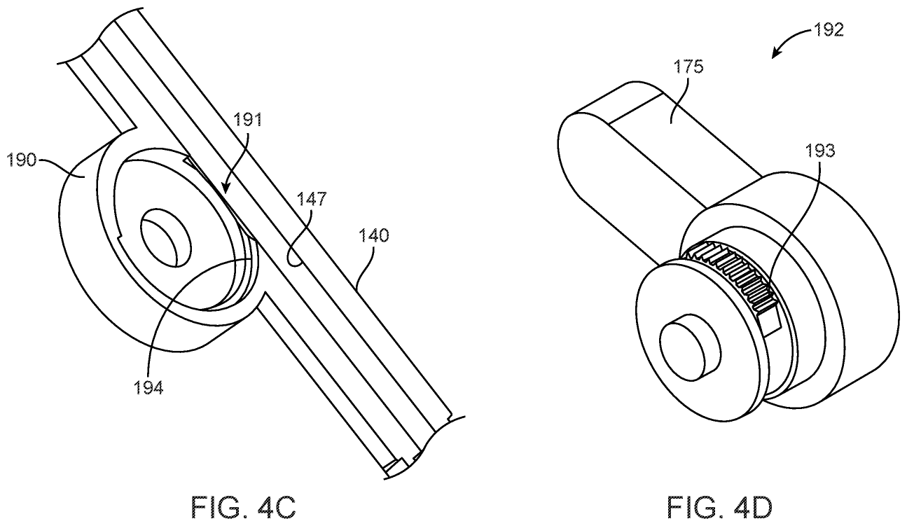

The actuator 175 can be a handle projecting from a cam body 192 positioned within a cam hub 190 coupled to a region of the sheath hub 140. FIGS. 4A-4C show the sheath hub 140 coupled to the cam hub 190 configured to receive the cam body 192, which is shown in FIG. 4D. The cam body 192 can incorporate a plurality of teeth 193 positioned on an outer perimeter of a portion of the cam body 192 creating an elliptical shape to the cam body 192. The cam body 192 can be received within the interior of the cam hub 190. The cam hub 190 can include an open cut 191 so that the interior of the cam hub 190 communicates and intersects with the sheath hub lumen 147 (see FIG. 4C). This allows for the plurality of teeth 193 on the cam body 192 to project through the open cut 191 into at least a portion of the sheath hub lumen 147. The push rod 150 can incorporate one or more surface features sized and spaced to engage with the teeth 193 of the cam body 192. The push rod 150 (or a hypotube coupled to the push rod 150) extending through the lumen 147 of the sheath hub 140 can come into contact with the teeth 193 within the region of the lumen 147 where it intersects the interior of the cam hub 190 (see FIG. 4C). The cam body 192 need not incorporate teeth 193 that engage with the push rod 150. The elliptical shape alone of the cam body 192 can be sufficient to pinch the push rod 150 against the lumen 147 of the sheath hub 140 to effect a lock and/or drive the push rod 150 forward upon rotation of the cam body 192.

As the cam body 192 is rotated around axis A′ relative to the hub 190, the region of the cam body 192 having the teeth 193 (or portion of the cam body 192 having the decreased cam radius) enters and travels distally through the sheath hub lumen 147 before exiting the sheath hub lumen 147 and entering the interior of the cam hub 190 once again. The teeth 193 can be received within a channel or groove 194 on the internal surface of the cam hub 190 (see FIGS. 4A-4C). The groove 194 within the cam hub 190 can extend around the full circumference of the hub 190 so that the cam body 192 can rotate the full 360 degrees relative to the cam hub 190. In other implementations, the groove 194 extends a shorter distance around the circumference so that the end of the groove 194 forms a stop to cam body rotation along a particular direction. The groove 194 can extend 90 degrees, 120 degrees, 150 degrees, 180 degrees, 210 degrees, and anywhere in between, along an inner surface of the cam hub 190 so that the cam body 192 rotates only a set number of degrees relative to the hub 190 before it hits the stop.

FIGS. 5A-5C show the toggling of the actuator 175 relative to the sheath hub lumen 147 between a first “locked” position, a second “released” position, and a third “deployment” position. FIG. 5A shows the actuator 175 fully cammed over so that the push rod 150 is pinched between the teeth 193 of the cam body 192 and the lumen 147 thereby effecting a lock. A user can urge the cam body 192 to rotate inside the cam hub 190 around axis A′ by toggling the actuator 175 away from the first “locked” position as shown in FIG. 5B. This “released” position eliminates any stiction between the sheath 130 and the device 105 being deployed because rotation of the actuator 175 around the axis A′ drives the push rod 150 forward a set distance, assuming the push rod 150 is not pinned, by the teeth 193 or decreased cam radius portion and allows the push rod 150 to move off of the lumen 147 wall. Alternatively, the teeth 193 of the cam body 192 engaged with the surface features of the push rod 150 can apply a force against the push rod 150, which is kept pinned. The friction between the teeth 193 on the cam body 192 and the surface features on the stationary push rod 150 urges the cam body 192 and attached sheath hub 140 to move in a proximal direction relative to the pinned push rod 150 as the actuator 175 is moved. Rotation of the cam body 192 is translated into axial retraction of the sheath hub 140 relative to the push rod 150.

As with the previous implementation, the distal end of the sheath hub 140 is connected to the outer sheath 130 such that the outer sheath 130 is retracted a fixed distance relative to the inner assembly (i.e., the push rod 150) in a controlled manner to break the built-up friction between the device 105 and the outer sheath 130. Once the greatest force is overcome in the initial stages of outer sheath withdrawal or the initial stages of push rod 150 advancement, which is relieved in a controlled manner by the release mechanism 170, the actuator 175 can be toggled fully to the “deployment” position (see FIG. 5C). In this “deployment” position, the teeth 193 enter the cam hub 190 such that the cam body 192 is fully withdrawn from the lumen 147 allowing free travel through the lumen 147, for example, so that longitudinal retraction of the outer sheath 130 can be performed per conventional pin-and-pull technique without risk of handle jerk or slippage or jumping of the stent out of the delivery system 100. One or more detents can be designed within the cam body 192 or cam hub 190 to provide tactile and/or audible feedback to a user regarding rotation and position of the actuator 175. The user depending on the number of clicks heard and/or felt can assess whether the actuator 175 is in the “locked”, “released”, or “deployment” position.

The release mechanism 170, regardless the specific configuration and whether the cam mechanism of FIGS. 3A-3B or the threaded knob of FIGS. 2A-2C is used, provides a mechanical advantage of initial sheath retraction that is controlled and limited to a fixed distance upon a single actuation of the actuator 175. The built-up friction and/or tension built up into the outer sheath 130 is released with the single actuation of the actuator 175 that is prior to deployment of the expandable device 105 with a second actuation, such as withdrawing the outer sheath 130 by pulling on the sheath hub 140 thereby providing a more accurate and controlled deployment. Thus, the release mechanism 170 described herein is not used to fully deploy the expandable device 105 (i.e., release the device 105 from its constrain allowing it to expand). Rather, the pin-and-pull mechanism can be used for full deployment after the release mechanism 170 described herein are used to initially release the friction and tension in the system 100. The release mechanism 170, once placed into the “unlocked” position, is then free from engagement with the push rod 150 so that the conventional pin-and-pull deployment mechanism can be used. In the implementation shown in FIGS. 2A-2C, after actuating the release mechanism 170, the push rod 150 can be held fixed and the outer sheath 130 manually retracted along the longitudinal axis A (e.g., by pulling on the sheath hub 140) relative to the fixed push rod 150 to deploy the expandable device 105 from its constraint within the outer sheath 130. In the implementation shown in FIGS. 3A-3B and 4A-4D, actuating the release mechanism 170 results in the cam body 192 rotating sufficiently around axis A′ so that the teeth 193 are received within the cam hub 190 and out of frictional engagement with the surface features of the push rod 150. Once the teeth 193 are out of engagement with the surface features of the push rod 150, the sheath hub 140 can be manually retracted in a proximal direction along the longitudinal axis A past the cam body 192 while the push rod 150 is held fixed to deploy the expandable device 105.

The components of the release mechanism 170 can be formed of industry standard materials, such as stainless steel and/or one or more polymers.

In aspects, description is made with reference to the figures. However, certain aspects may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “an aspect,” “one aspect,” “one implementation, “an implementation,” or the like, means that a particular feature, structure, configuration, or characteristic described is included in at least one embodiment, aspect, or implementation. Thus, the appearance of the phrase “one embodiment,” “an embodiment,” “one aspect,” “an aspect,” “one implementation, “an implementation,” or the like, in various placed throughout this specification are not necessarily referring to the same embodiment, aspect, or implementation. Furthermore, the particular features, structures, configurations, or characteristics may be combined in any suitable manner in one or more implementations.

The use of relative terms throughout the description may denote a relative position or direction or orientation and is not intended to be limiting. For example, “distal” may indicate a first direction away from a reference point. Similarly, “proximal” may indicate a location in a second direction opposite to the first direction. Use of the terms “front,” “side,” “back,” “bottom” and “top” as well as “anterior,” “posterior,” “caudal,” “cephalad” and the like or used to establish relative frames of reference, and are not intended to limit the use or orientation of any of the devices described herein in the various implementations.

The word “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples, embodiments, aspects, and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

What is claimed:

1. An endoluminal prostheses delivery system comprising:
an outer sheath comprising a distal end region, a proximal end region and a lumen extending along a longitudinal axis between the proximal end region and the distal end region;
an expandable device;
an inner member extending through the lumen of the outer sheath, at least a portion of the inner member configured to support the expandable device within the lumen near the distal end region of the outer sheath; and
a release mechanism configured to move at least one of the outer sheath and the inner member an initial distance in a controlled manner to break friction between the expandable device and the outer sheath prior to deployment of the expandable device from the lumen.

2. The delivery system of claim 1, wherein the release mechanism is configured to retract the outer sheath the distance.

3. The delivery system of claim 1, wherein the release mechanism is configured to advance the inner member the distance.

4. The delivery system of claim 1, wherein the distance is about 5 mm up to about 1 cm.

5. The delivery system of claim 1, wherein the release mechanism comprises an actuator configured to be toggled between at least a first position and at least a second position.

6. The delivery system of claim 5, wherein the actuator is toggled by rotation around longitudinal axis of the outer sheath.

7. The delivery system of claim 6, wherein the release mechanism further comprises a threaded internal component coupled to a proximal end region of the inner member, wherein the threaded internal component is in threaded engagement with a corresponding thread on an internal surface of the actuator.

8. The delivery system of claim 7, wherein toggling the actuator around the longitudinal axis from the first position to the second position while the inner member is held fixed and the outer sheath is not held fixed retracts the outer sheath proximally as the corresponding thread of the actuator translates along the threaded internal component.

9. The delivery system of claim 7, wherein toggling the actuator around the longitudinal axis from the first position to the second position while the proximal end region of the outer sheath is held fixed and the inner member is not held fixed advances the inner member distally as the threaded internal component translates along the corresponding thread of the actuator.

10. The delivery system of claim 7, wherein the actuator further comprises a projection on an outer surface of the actuator configured to be received against or within a first stop on a proximal end region of the outer sheath.

11. The delivery system of claim 10, wherein the first stop provides tactile and/or auditory feedback regarding position of the actuator relative to the first stop.

12. The delivery system of claim 10, wherein the actuator comprises a projection and the first stop comprises a first surface feature projecting a distance radially outward from an outer surface of the outer sheath and a second surface feature projecting a second distance radially outward from the outer surface of the outer sheath, the first surface feature projecting further than the second surface feature such that the projection on the actuator can slide over the second surface feature and is prevented from sliding over the first surface feature.

13. The delivery system of claim 12, wherein receipt of the projection between the first and second surface features provides tactile and/or auditory feedback regarding position of the actuator relative to the first stop.

14. The delivery system of claim 12, further comprising one or more markings on the outer surface of the outer sheath relative to the first stop providing information regarding actuator position.

15. The delivery system of claim 10, further comprising a second stop on the proximal end region of the outer sheath located less than 360 degrees around the longitudinal axis relative to the first stop.

16. The delivery system of claim 5, wherein the actuator is toggled by rotation around an axis orthogonal to the longitudinal axis of the outer sheath.

17. The delivery system of claim 16, wherein the release mechanism further comprises a cam body positioned within a cam hub coupled to a proximal end region of the outer sheath.

18. The delivery system of claim 17, wherein the cam body has an elliptical shape configured to project through an opening in the cam body and into the lumen of the proximal end region of the outer sheath.

19. The delivery system of claim 18, wherein the elliptical shape of the cam body comprises a plurality of teeth.

20. The delivery system of claim 19, wherein the inner member includes one or more surface features sized and spaced to engage with the plurality of teeth on the cam body.

21. The delivery system of claim 18, wherein the cam body and the inner member come into contact with one another within a region of the lumen of the outer sheath.

22. The delivery system of claim 21, wherein the cam body pinches the inner member against an inner wall of the outer sheath to lock and/or drive the inner member upon rotation of the cam body.

23. The delivery system of claim 21, wherein toggling the actuator comprises moving the actuator relative to the lumen between a first locked position, a second released position, and a third deployment position.

24. The delivery system of claim 23, the first locked position comprises the actuator fully cammed over so that the inner member is pinched between the cam body and the lumen effecting a lock.

25. The delivery system of claim 24, wherein rotating the cam body around the axis away from the first locked position toward the second released position while the inner member is not fixed drives the inner member forward the distance.

26. The delivery system of claim 24, wherein rotating the cam body around the axis away from the first locked position toward the second released position while the inner member is kept fixed retracts the outer sheath the distance in a proximal direction relative to the inner member.

27. The delivery system of claim 23, wherein rotating the cam body around the axis away from the second released position toward the third deployment position fully withdraws the cam body from the lumen of the outer sheath.

28. The delivery system of claim 23, wherein the cam body and/or the cam hub incorporates one or more detents to provide tactile and/or audible feedback regarding relative position of the actuator.
